(19) United States
(12) Patent Application Publication
(10) Pub. No.: US 2009/0263513 A1
(43) Pub. Date: Oct. 22, 2009

(54) COSMETIC SKIN LIGHTENING FORMULATION

(76) Inventor: Jan Marini, San Jose, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/106,054

(22) Filed: Apr. 18, 2008

(51) Int. Cl. A61K 36/82; A61K 31/351; A61K 31/07; A61K 36/00; A61Q 19/00; A61K 31/05; A61K 31/60

(52) U.S. Cl. 424/729; 514/460; 514/725; 514/731; 514/165; 424/773

(57) ABSTRACT

The present invention features novel cosmetic skin care compositions for lightening hyperpigmentation.

### BACKGROUND OF THE INVENTION

Many people are concerned with the degree of pigmentation of their skin. For example, people with age spots or freckles may wish such pigmented spots to be less pronounced. Others may wish to reduce the skin darkening caused by exposure to sunlight or to lighten their natural skin color. Skin may appear lighter or darker than normal in concentrated areas. Such skin pigmentation disorders occur because the body produces too much or too little melanin, which is the pigment produced by melanocytes in the skin.

Melanin is the brown to black pigment of hair and skin. The brown-black eumelanins are formed predominantly from hydroxy-substituted aromatic amino acids such as L-tyrosine and L-DOPA, and the yellow to red pheomelanins are additionally formed from sulfur-containing molecules. Starting from L-tyrosines, the copper-containing key enzyme tyrosinase forms L-3,4-dihydroxyphenylalanine (L-DOPA), which for its part is oxidized again by the tyrosinase via the red-brown dopaquinone to give melanin.

Melanosomes are organelles found in melanocytes, a cell type present at the dermis-epidermis junction. In the melanosomes, melanin is polymerized from monomers, and is transferred to the neighboring keratinocytes. The keratinocytes divide and differentiate and thus transport the melanosome to the surface of the skin. The intensity of the skin color is directly related to the number, the size, the melanin content, dispersion of melanin, the rate of formation and migration/transfer of melanosomes to the keratinocytes. There have been reports wherein inhibitors of tyrosinase such as hydroquinone and its derivatives, kojic acid, catechols, mercaptoamines, alpha hydroxy acids and others have been used in cosmetic compositions to regulate skin pigmentation.

If the melanin-forming melanocytes are not distributed evenly in the human skin, pigmentation spots form, which are either lighter or darker than the surrounding areas of skin. Increased melanin production, also known as hyperpigmentation, is often referred to as melasma, chloasma or solar lentigenes. Melasma is a general term describing darkening of the skin. Chloasma is generally used to describe skin discolorations caused by hormones. These hormonal changes are usually the result of pregnancy, birth control pills or estrogen replacement therapy. Solar lentigenes refers to darkened spots on the skin caused by the sun. These spots are quite common in adults with a long history of unprotected sun exposure. The most common cause of darkened areas of skin, brown spots or areas of discoloration is unprotected sun exposure, although hyperpigmentation can be caused by skin damage, such as blemishes, wounds or rashes.

Solar lentigenes can emerge as small- to medium-sized brown patches of freckling that grow and accumulate over time on areas of the body that receive the most unprotected sun exposure, such as the back of the hands, forearms, chest, and face. For those with darker skin colors, these discolorations can appear as patches or areas of ashen-gray skin.

Therefore, there is a continuing need for new skin lightening agents, with improved overall effectiveness. The compositions and their methods of use presented herein meet that need.

### PUBLICATIONS

U.S. Patents relating to skin lightening formulations include U.S. Pat. No. 7,332,152, “Cosmetic composition”; U.S. Pat. No. 7,300,646 “Skin lightening agents, compositions and methods”; U.S. Pat. No. 7,288,263 “Compositions and methods for treatment of skin discoloration”; U.S. Pat. No. 7,282,592 “Resorcinol derivatives”; U.S. Pat. No. 6,939,552 “Dual compartment packaged cosmetic composition”; U.S. Pat. No. 6,521,267 “Tyrosinase inhibitors from plants”; U.S. Pat. No. 6,497,860 “Skin lightening compositions”; U.S. Pat. No. 6,436,378 “Composition”; U.S. Pat. No. 6,110,448 “Method for causing skin lightening”; U.S. Pat. No. 6,066,312 “Topical composition for application to the skin containing an ellagic acid-based compound or salt thereof”; U.S. Pat. No. 6,057,360 “Compositions and systems for the treatment of hyperpigmentation”; U.S. Pat. No. 5,989,576 “Method of lightening skin”; U.S. Pat. No. 5,545,399 “Cosmetic composition”; U.S. Pat. No. 5,279,834 “Pharmaceutical or cosmetic composition containing hydroquinone and kojic acid”.

### SUMMARY OF THE INVENTION

The present invention features cosmetic skin care compositions for reducing hyperpigmentation of the skin. Specifically, the skin care compositions presented herein contain a synergistic combination of: a stable kojic acid derivative, a retinoid, and a resorcinol derivative, for use in improving the appearance of hyperpigmented regions of the skin. The skin care compositions of the invention may further comprise alpha-hydroxy, beta-hydroxy, or dicarboxylic acids, including lactic acid, glycolic acid, azelaic acid, salicylic acid, and the like. The formulation is provided in a cosmetically acceptable vehicle(s), which may further comprise skin soothing/conditioning agents, and permeation enhancers, e.g. transcutol. Accordingly, the synergistic combinations of the active ingredients of the invention are formulated as skin care cosmetic compositions that can be applied directly to the skin so as to lighten the skin.

According to the first aspect of the invention, there is provided a synergistic skin lightening composition comprising: i. 0.1-10% by weight a stable kojic acid derivative; ii. 0.1-5% by weight a retinoid; and iii. 0.1-5% by weight of resorcinol or a derivative thereof. In one embodiment of the invention, the kojic acid derivative is kojic dipalmitate. In one embodiment, the resorcinol or derivative is 4-hexylresorcinol. In one embodiment the retinoid is retinol.

In the second aspect of the invention, a method is provided for lightening hyperpigmentation of the skin, the method comprising applying topically a cosmetic formulation with a synergistic skin lightening composition comprising: i. 0.1-10% by weight a stable kojic acid derivative; ii. 0.1-5% by weight a retinoid; and iii. 0.1-5% by weight of resorcinol or a derivative thereof. The composition is topically administered as a lotion or cream for a period of time sufficient to accomplish the desired lightening effect. In some embodiments the composition is administered once daily, or twice daily, and for at least about one week, at least about two weeks, at least about one month, or longer as desired,

### DETAILED DESCRIPTION OF THE EMBODIMENTS

Topical compositions are provided for the lightening of skin hyperpigmentation, which includes a variety of dark regions on the skin. Age spots are a common form of hyperpigmentation. They occur due to sun damage, and may be referred to as solar lentigenes. These small, darkened patches are usually found on the hands and face or other areas frequently exposed to the sun. Melasma or chloasma spots are similar in appearance to age spots but are larger areas of darkened skin that appear most often as a result of hormonal changes. Pregnancy, for example, can trigger overproduction of melanin that causes the “mask of pregnancy” on the face and darkened skin on the abdomen and other areas. Women who take birth control pills may also develop hyperpigmentation because their bodies undergo similar kind of hormonal changes that occur during pregnancy. If one is really bothered by the pigment, the birth control pills should be stopped. Changes in skin color can result from outside causes. For example, skin diseases such as acne may leave post-inflammatory hyperpigmentation or flat macule pigmentation after the condition clears. Other causes of dark spots are injuries to the skin, including some surgeries. Freckles are small brown spots that can appear anywhere on the body, but are most common on the face and arms.

Accordingly, the compositions of the invention may be topically applied to any subject and used to treat a variety of skin hyperpigmentation conditions, including but not limited to: solar lentigenes, melasma, chloasma, scars, freckles, and other local hyperpigmented regions of the skin. The compositions may also be topically applied to treat fine lines and wrinkles and improve skin texture.

The compositions of the invention provide for complementary and synergistic mechanisms of lightening, where kojic acid and hexylresorcinol act to block copper metabolism and inhibit production of tyrosinase, and retinol inhibits melanin deposition in the skin. Anti-inflammatory in the formulation, such as green tea and the like help to reduce the “reactive” effect of melanocytes. By blocking multiple pathways, a greater efficacy is achieved in lightening. In addition to skin lightening properties, the compositions of the invention provide anti-aging benefits.

### Components of the Cosmetic Compositions

Kojic acid. The compositions of the invention comprise a stable kojic acid derivative, usually a fatty acid ester of kojic acid, having the structure:

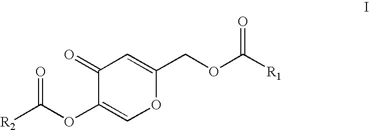

where R₁ and R₂ are independently selected from C1-C22 alkyls, having not more than 1, usually 0 unsaturated bonds. Fatty acids of interest for esterification include palmitic acid, palmitoleic acid, oleic acid, linoleic acid, linolenic acid, and the like. In some preferred embodiments the kojic acid derivative is kojic dipalmitate, shown below.

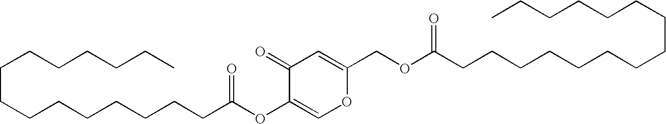

Cosmetic formulation of the present invention comprise at least about 0.1% by weight of a stable kojic acid derivative, usually at least about 0.5%, more usually at least about 1%, and preferably from about 2.5% to about 5%, usually not more than about 10%.

Retinoid. Retinol (vitamin A), is a fat-soluble vitamin. It belongs to the family of chemical compounds known as retinoids. Derivatives include retinal, also known as retinaldehyde, which can be reversibly reduced to produce retinol or it can be irreversibly oxidized to produce retinoic acid (tretinoin, isotretinoin). The best described active retinoid metabolites are 11-cis-retinal and the all-trans and 9-cis-isomers of retinoic acid. Retinol may be measured-in international units (IU), where 1 IU of retinol is equivalent to approximately 0.3 micrograms (300 nanograms).

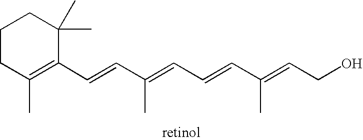

Cosmetic formulation of the present invention comprise at least about 0.1% by weight of a retinoid, usually at least about 0.5%, more usually at least about 0.75%, and preferably from about 1.5% to about 2.5%, e.g. about 2%, and usually not more than about 5%. A preferred compound for the compositions of the present invention is retinol.

Resorcinol. Resorcinol is the 1,3-isomer of benzenediol. Resorcinol derivatives have cosmetic skin and hair benefits, particularly 4-substituted resorcinol derivatives. Resorcinol derivatives are described in many publications, including U.S. Pat. No. 4,959,393; U.S. Pat. No. 6,132,740; and U.S. Pat. No. 6,504,037. Resorcinol derivatives are known compounds and can be readily obtained by various means. Resorcinol derivatives of interest for the present invention include those of the formula:

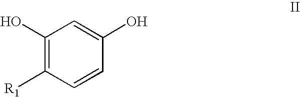

where R₁ is an alkyl, usually a C1 to C12 alkyl, more usually a C6 to C12 alkyl.

In some embodiments of the invention the resorcinol derivative is hexylresorcinol:

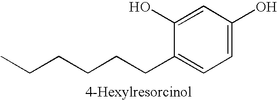

Cosmetic formulation of the present invention comprise at least about 0.1% by weight of a resorcinol derivative, usually at least about 0.5%, and preferably from about 0.75% to about 2%, e.g. about 1%, and usually not more than about 5%. Preferred compounds for the compositions of the present invention are hexylresorcinol and dodecyiresorcinol.

### Additional Agents

In addition to the combination of kojic acid derivative, retinoid, and resorcinol derivative, the cosmetic formulation may include other active agents. In some embodiments, the formulation comprises permeation enhancer, e.g. transcutol, (diethylene glycol monoethyl ether), which may be provided at a weight/weight concentration of from about 0.1% to about 10%, usually from about 2.5% to about 7.5%, more usually about 5%.

Salicylic acid is an organic carboxylic acid that is produced in plants. Salicylic acid can act as an oxygen trap and thereby initiate free radical reactions. When applied topically to the skin it additionally promotes the sloughing off of dead skin cells and can thereby help prevent the pores from clogging up. Accordingly, salicylic acid can act synergistically in a combination formulation with the other agents of the of the invention to effectively treat against a number of skin conditions, such as acne, psoriasis, and the like. The cosmetic compositions of the present invention may contain salicylic acid in amounts that are safe and effective, for instance, at concentrations of at least about 0.5%, at least about 1%, around about 2%, at and usually not more than about 5% (weight/weight).

Azelaic acid is a saturated dicarboxylic acid that is produced by Malassezia furfur, a yeast that lives on normal skin. When applied topically to the skin in a combination formulation it acts synergistically with the other agents of the of the invention. The cosmetic compositions of the present invention may contain azelaic acid in amounts that are safe and effective, for instance, at concentrations of at least about 0.5%, at least about 1%, around about 2%, at and usually not more than about 5% (weight/weight).

Glycolic acid and lactic acid are α-hydroxy acids that is produced in various fruits and vegetables. Glycolic acid can act synergistically with the other ingredients of the invention to help them deeply penetrate the skin and can thereby help improve the skins appearance and texture. Once applied to the skin glycolic acid can react with the upper layer of the epidermis thereby weakening the binding properties of the lipids that hold the dead skin cells together. This promotes the sloughing off of the outer skin cells so as to reveal the underlying, healthier, smoother, brighter-looking skin underneath. The cosmetic compositions of the present invention may contain glycolic acid in amounts that are safe and effective, for instance, at concentrations of at least about 0.5%, at least about 1%, around about 2%, at and usually not more than about 5% (weight/weight).

Licorice extract is an lipophilic extract obtained from the roots of Glycyrrhiza glabra L. (syn. Liquiritae officinalis Moench., Fam. Fabaceae) that is useful as a cosmetic ingredient. Its skin whitening, anti-inflammatory, antimicrobial and antioxidant properties makes it an useful ingredient in topical applications. The cosmetic compositions of the present invention may contain licorice extract in amounts that are safe and effective, for instance, at concentrations of at least about 0.01%, at least about 0.5%, and usually not more than about 1% (weight/weight).

Green tea extract is derived from leaves of Camellia sinensis, the plant from which green, black and oolong teas are made. The active ingredients in green tea extract are polyphenols in the form of flavinoids like catechins and epigallocatechin gallate (EGCG). The cosmetic compositions of the present invention may contain green tea extract in amounts that are safe and effective, for instance, at concentrations of at least about 0.01%, at least about 0.5%, and usually not more than about 1% (weight/weight).

Peptides, including but not limited to, di-, tri-, tetra-, and pentapeptides, as well as oligo-peptides of from about 6 to about 30 amino acids in length and derivatives thereof, may be included as cosmetic benefit agents of the present invention in amounts that are safe and effective. Such peptides are usually acylated, comprising at least one lipid moiety, which moiety may be myristoyl, palmitoyl, etc., which increases the hydrophobicity of the peptide. Myristoyl or palmitoyl pentapeptides are of particular interest, as is the use of thymosin β4. As used herein, “peptides” refers to both naturally occurring peptides and synthesized peptides. Below is a non-limiting list of exemplary peptide agents that find use in the cosmetic compositions of the present invention.

Certain peptide agents of interest stimulate the macromolecules of the dermis. For example, synthetic peptides such as lamin, biopeptide CL or paimitoyloligopeptide (SEDERMA) activate the synthesis of collagen. In addition, natural peptides extracted from plants, such as the soya bean hydrolyzate marketed by the company COLETICA under the trademark Phytokine™, also provide this activity.

Certain other peptide agents act on the synthesis of fibronectin, such as the palmitoyl pentapeptide marketed by the company SEDERMA under the trademark Matrixil™. Still other of these peptides inhibit metalloproteinases, such as oligopeptides and lipopeptides, lipoamino acids, and malt extract marketed by the company COLETICA under the trademark Collalift™, while some peptide agents inhibit serine proteases, such as leukocyte elastase or cathepsin G, including the peptide extract of seeds of leguminous plants (Pisum sativum) which is marketed by the company LSN under the trademark Parelastyl™ and certain pseudo-dipeptides.

Some peptide agents that find use in the cosmetic compositions of the present invention stimulate the proliferation of fibroblasts, including plant polypeptides (or extracts) from soya bean (e.g., Eleseryl SH-VEG 8™ marketed by the company LSN, or Raffermine™ marketed by the company SILAB).

In addition to the activities noted above, certain peptide agents may provide anti-bacterial or anti-fungal activity to the cosmetic compositions of the invention. For example, see U.S. Pat. No. 6,835,536, which describes numerous cationic antimicrobial peptides (e.g. indolicidins or analogs or derivatives thereof derived from natural sources or produced synthetically) that find use in topically-applied compositions for the treatment of acne. Similarly, U.S. Pat. No. 6,713,078 described granulysin peptides useful in the topical treatment of acne.

The peptide agents of the present invention are formulated at an effective concentration within the subject cosmetic compositions, meaning at a concentration that provides the intended benefit when applied topically. An effective concentration of peptide or peptide-like compounds is preferably in a range of at least about 0.5%, more usually at least about 1.0%, at least about 2.5%, at least about 5%, and less than about 50% by weight, usually less than about 10% by weight, or less than about 5%.

### Skin Soothing/Conditioning Agents

The cosmetic compositions of the present invention may contain agents that sooth, condition and/or heal the skin and hair. One such agent is panthenol, a pro-vitamin moisturizing agent related to Vitamin E. Panthenol is easily incorporated into cosmetic formulations and readily penetrates the skin. Panthenol derivatives (e.g., ethyl panthenol) also find use in the compositions of the invention as do agents such as aloe vera, pantothenic acid and its derivatives, allantoin, bisabolol, and dipotassium glycyrrhizinate. One or more of these agents may be provided in an amount from about 1 to about 25% by weight. Many other skin conditioning/soothing agents can be included in the subject compositions, some of which are discussed below.

The compositions of the invention may optionally comprise other skin benefit materials. These include estradiol; progesterone; pregnanalone; coenzyme Q10; methylsolanomethane (MSM); copper peptide (copper extract); plankton extract (phytosome); kojic acid; ascorbyl palmitate; all-trans-retinol; broparoestrol; estrone; adrostenedione; androstanediols; etc. The steroids will generally be present at a concentration of less than about 5% or about 10% of the total by weight of the composition, while the other skin benefit materials may be present at higher levels, for example as much as about 10 to about 15%.

The compositions may further comprise sunscreens to lower skin's exposure to harmful UV rays. Sunscreens include those materials commonly employed to block ultraviolet light. Illustrative compounds are the derivatives of PABA, cinnamate and derivatives of salicylate (other than ferulyl salicylate). For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. Dermascreen may also be used. The exact amount of sunscreen employed in the compositions can vary depending upon the degree of protection desired from the sun's UV radiation.

The amounts of cosmetic or dermatological auxiliaries and additives and perfume to be used in each case can easily be determined by simple exploratory experiments by the person skilled in the art, depending on the nature of the product in question.

### Cosmetically Acceptable Vehicle

The compositions of the invention include a cosmetically acceptable vehicle to act as a diluent, dispersant or carrier for the skin lightening agents, so as to facilitate distribution and uptake when the composition is applied to the skin. Vehicles other than or in addition to water can include liquid or solid emollients, solvents, humectants, thickeners and powders.

The cosmetically acceptable vehicle will usually form 5% to 99.9%, preferably from 25% to 80% by weight of the composition, and can, in the absence of other cosmetic adjuncts, form the balance of the composition.

The compositions may be in the form of aqueous, aqueous/alcoholic or oily solutions; dispersions of the lotion or serum type; anhydrous or lipophilic gels; emulsions of liquid or semi-liquid consistency, which are obtained by dispersion of a fatty phase in an aqueous phase (O/W) or conversely (W/O); or suspensions or emulsions of smooth, semi-solid or solid consistency of the cream or gel type. These compositions are formulated according to the usual techniques as are well known to this art.

When the compositions of the invention are formulated as an emulsion, the proportion of the fatty phase may range from 5%.to 80% by weight, and preferably from 5% to 50% by weight, relative to the total weight of the composition. Oils, emulsifiers and co-emulsifiers incorporated in the composition in .emulsion form are selected from among those used conventionally in the cosmetic or dermatological field. The emulsifier and co emulsifier may be present in the composition at a proportion ranging from 0.3% to 30% by weight, and preferably from 0.5% to 20% by weight, relative to the total weight of the composition.

When the compositions of the invention are formulated as an oily solution or gel, the fatty phase may constitute more than 90% of the total weight of the composition.

The compositions of the invention may be in the form of body cleansing compositions. As such, these compositions may contain at least one wash-active surfactant in an aqueous base are preferred embodiments of the invention. The surfactants can be present, alone or in a mixture, and are contained in an amount of preferable from 1 to 50% by weight, especially preferably from 1 to 30% by weight. Nonionic surfactants, amphoteric surfactants, zwitterionic surfactants and anionic surfactants are generally suitable.

Suitable anionic surfactants include, e.g. alkaline or alkaline earth salts, alpha-olefin sulfonates, sulfosuccinates, disodium laureth-3 sulfosuccinate, disodium PEG-5 lauryl citrate sulfosuccinate, disodium ricinolamido MEA-sulfosuccinate or disddium laurylamido MEA-sulfosuccinate and alkyl ether carboxylates.

Suitable nonionic surfactants include e.g. alkoxylated fatty alcohols, alkoxylated fatty acid esters, alkoxylated partial glycerides, saturated or unsaturated fatty acids, alkoxylated polyol esters, and alkylpolyglucosides, such as coconut glucosides, lauryl glycosides or decylglucosides. For example, ethoxylated lauryl alcohol, tetradecyl alcohol, cetyl alcohol, oleyl alcohol or stearyl alcohol, which are used alone or in mixtures with each other, as well as fatty alcohols of ethoxylated lanolin, are suitable as fatty alcohol ethoxylates. Furthermore the ethoxylated fatty acid sugar esters known as nonionic surfactants, especially ethoxylated sorbitan fatty acid ester, are suitable for use in the cosmetic preparations according to the invention. The suitable ethoxylated fatty acid sugar esters include those marketed under the trade names Tween™ and Arlacel™ by ICI surfactants and the alkyl-polyglycosides, which are marketed under the trade names Plantaren™ or Plantacare™ by Henkel or under the trade name Oramix™ by Seppic.

Suitable amphoteric surfactants include for example betaines, such as cocoamidopropylbetaine or lauryl betaine, sulfobetaines, such as cocoamidopropyl hydroxysultaine, glycinates, such as cocoamphoglycinate (INCI-name: sodium cocoamphoacetate) and diglycinates and propionates, such as cocoampho-propionate.

The compositions of the invention may also contain additives and adjuvants which are conventional in the cosmetic, pharmaceutical or dermatological field, such as hydrophilic or lipophilic gelling agents, hydrophilic or lipophilic active agents, preservatives, antioxidants, solvents, fragrances, fillers, bactericides, odor absorbers and dyestuffs or colorants. The amounts of these various additives and adjuvants are those conventionally used in the field, and, for example, range from 0.01% to 10% of the total weight of the composition. Depending on their nature, these additives and adjuvants may be introduced into the fatty phase or into the aqueous phase.

Exemplary oils which may be used according to this invention include mineral oils (liquid petrolatum), plant oils (liquid fraction of karite butter, sunflower oil), animal oils (perhydrosqualen(e), synthetic oils (purcellin oil), silicone oils (cyclomethicone) and fluoro oils (perfluoropolyethers). Fatty alcohols, fatty acids (stearic acid) and waxes (paraffin wax, carnauba wax and beeswax) may also be used as fats.

Emulsifiers which may be used include glyceryl stearate, polysorbate 60, PEG-6/PEG-32/glycol stearate mixture, etc. Solvents which may be used include the lower alcohols, in particular ethanol and isopropanol, and propylene glycol.

Hydrophilic gelling agents include carboxyvinyl polymers (carbomer), acrylic copolymers such as acrylate/alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose and polyethylene.

An oil or oily material may be present, together with an emollient to provide either a water-in- oil emulsion or an oil-in-water emulsion, depending largely on the average hydrophilic-lipophilic balance (HLB) of the emollient employed. Levels of such emollients may range from about 0.5% to about 50%, preferably between about 5% and 30% by weight of the total composition. Emollients may be classified under such general chemical categories as esters, fatty acids and alcohols, polyols and hydrocarbons.

Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, oleyl eurcate and stearyl oleate. Preferred esters include coco-caprylate/caprate (a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids.

Among the polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol are also especially preferred as penetration enhancers.

Exemplary hydrocarbons which may serve as emollients are those having hydrocarbon chains anywhere from 12 to 30 carbon atoms. Specific examples include mineral oil, petroleum jelly, squalene and isoparaffins.

Another category of functional ingredients within the cosmetic compositions of the present invention are thickeners. A thickener will usually be present in amounts anywhere from 0.1 to 20% by weight, preferably from about 0.5% to 10% by weight of the composition. Exemplary thickeners are cross-linked polyacrylate materials available under the trademark Carbopol. Gums may be employed such as xanthan, carrageenan, gelatin, karaya, pectin and locust beans gum. Under certain circumstances the thickening function may be accomplished by a material also serving as a silicone or emollient. For instance, silicone gums in excess of 10 centistokes and esters such as glycerol stearate have dual functionality.

Powders may be incorporated into the cosmetic composition of the invention. These powders include chalk, talc, kaolin, starch, smectite clays, chemically modified magnesium aluminum silicate, organically modified montmorillonite clay, hydrated aluminum silicate, fumed silica, aluminum starch octenyl succinate and mixtures thereof.

Other adjunct minor components may also be incorporated into the cosmetic compositions. These ingredients may include coloring agents, opacifiers and perfumes. Amounts of these other adjunct minor components may range anywhere from 0.001% up to 20% by weight of the composition.

Accordingly, a composition of the invention comprises a retinoid, a stable kojic acid derivative, and a resorcinol derivative, which may be a synergistic combination, and optionally in combination with one or more of a permeation enhancer, an azelaic acid or a derivative thereof, salidylic acid or a derivative thereof, glycolic acid or a derivative thereof, licorice extract, and green tea extract, and/or a cosmetically acceptable vehicle. Furthermore, a composition of the invention may include additional agents or additives that are not in themselves active agents but play a role in promoting the usefulness or effectiveness of an active agent.

Compositions of the invention may be applied to any subject and used to treat a variety of hyperpigmentation conditions, e.g. solar lentigenes, melasma, chloasma, scars, freckles, and other local hyperpigmented regions of the skin. A typical composition of the invention is formulated as a solution, lotion, cream, gel, ointment, liniment, solvent, emulsion, dispersion, hydrodispersion, aerosol, propellant, soap, exfoliant or transdermal patch, which may be applied topically to the skin so as to treat, prevent, wash, condition or otherwise effect a condition of the skin.

### Product Use, Form, and Packaging

In use, a quantity of the composition, for example from 1 to 100 ml, is applied to a site of interest from a suitable container or applicator and, if necessary, it is then spread over and/or rubbed into the site using the hand or fingers or a suitable device. The product may be specifically formulated for use as a treatment for a specific area, e.g. the hands, the face, the arms, etc.

The cosmetic composition of the invention can be formulated in any form suitable for application to the site of interest, including a lotion, cream, gel, or the like. The composition can be packaged in any suitable container to suit its viscosity and intended use by the consumer. For example, a lotion or cream can be packaged in a bottle, or a propellant-driven aerosol device or a container fitted with a pump suitable for finger operation. When the composition is a cream, it can simply be stored in a non-deformable bottle or squeeze container, such as a tube or a lidded jar. The invention accordingly also provides a closed container containing a cosmetically acceptable composition as herein defined.

The following examples are put forth so as to provide those of ordinary skill in the art with a complete disclosure and description of how to make and use the subject invention, and are not intended to limit the scope of what is regarded as the invention. Efforts have been made to insure accuracy with respect to the numbers used (e.g. amounts, temperature, concentrations, etc.) but some experimental errors and deviations should be allowed for. Unless otherwise indicated, parts are parts by weight, molecular weight is weight average molecular weight, temperature is in degrees centigrade, and pressure is at or near atmospheric.

### EXAMPLE 1

Example 1 illustrates topical compositions according to the present invention. The compositions can be processed in conventional manner. They are suitable for cosmetic use. In particular the compositions are suitable for application to a site of interest for the treatment of a variety of skin hyperpigmentation conditions or disorders, e.g. solar lentigenes, melasma, chloasma, scars, freckles, and other local hyperpigmented regions of the skin.

LIGHTENING LOTION

| RM NAME | % (w/w) |
|---|---|
| D.I.WATER | q.s. to 100% |
| KOJIC DIPALMITATE | 5.00 |
| TRANSCUTOL | 5.00 |
| BUTYLENE GLYCOL | 4.00 |
| STEARYL ALCOHOL | 3.00 |
| C12-C15 ALKYL BENZOATE | 3.00 |
| SALICYLIC ACID | 2.00 |
| CAPRIC/CAPRYLIC TRIGLYCERIDE | 2.00 |
| CETYL ALCOHOL | 2.00 |
| MODIFIED POTATO STARCH | 2.00 |
| RETINOL 10S (0.2% ON ACTIVE BASIS) | 2.00 |
| HEXYLRESORCINOL | 1.00 |
| GLYCERIN | 1.00 |
| DIMETH1CONE | 1.00 |
| PROPYLENE GLYCOL, DIAZOLIDINYL UREA, | 1.00 |
| METHYLPARABEN, PROPYLPARABEN | |
| GLYCERIN, PROPYLENE GLYCOL, GLYCERYL | 1.00 |
| ACRYLATE/ACRYLIC ACID COPOLYMER | |
| GLYCERIN, PROPYLENE GLYCOL, CUCUMIS | 1.00 |
| SATIVUS(CUCUMBER)FRUIT EXTRACT, ARNICA | |
| MONTANA FLOWER EXTRACT, ANACYCLUS | |
| PYRETHRUM ROOT EXTRACT | |
| HYDROXYETHYLCELLULOSE(AND) SODIUM | 0.80 |
| ACETATE (AND) WATER | |
| EMULSIFYING WAX | 0.50 |
| POLYSORBATE 60 | 0.50 |
| LINSEED OIL, PALM OIL AMINOPROPANEDIOL | 0.10 |
| ESTERS | |
| JAPANESE GREEN TEA EXTRACT | 0.10 |
| DL-ALPHA TOCOPHERYL ACETATE | 0.10 |
| DIPOTASSIUM GYLCYRRHIZATE | 0.01 |
| TOTAL | 100.00% |

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A skin lightening cosmetic composition for topical application comprising:
from about 0.1 to about 10% by weight a stable kojic acid derivative;
from about 0.1 to about 5% by weight retinoid:
from about 0.1 to about 5% by weight of a resorcinol derivative; and
a cosmetically acceptable vehicle.

2. The skin lightening composition of claim 1, wherein the stable kojic acid derivative has the structure: 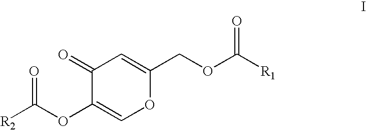
where R₁ and R₂ are independently selected from C1-C22 alkyls, having not more than 1, unsaturated bonds.

3. The skin lightening composition of claim 2, wherein the stable kojic acid derivative is kojic dipalmitate at a concentration of from about 2.5% to about 5%.

4. The skin lightening composition of claim 1, wherein the retinoid is retinol.

5. The skin lightening composition of claim 4, wherein retinol is present at a concentration of about 1.5% to about 2.5%.

6. The skin lightening composition of claim 1, wherein the resorcinol derivative has the structure: 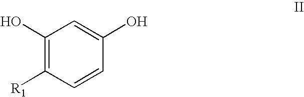
where R₁ is a C1 to C12 alkyl.

7. The skin lightening composition of claim 6, wherein the resorcinol derivative is 4.-hexylresorcinol at a concentration of from about 0.75% to about 2%.

8. The skin lightening composition of claim 1, wherein the stable kojic acid derivative is kojic dipalmitate at a concentration of 2.5% to 5%, the retinoid is retinol at a concentration of.5% to 2.5%; and the resorcinol derivative is 4-hexylresorcinol at a concentration of 0.75% to 2%.

9. The skin lightening composition of claim 8, further comprising salicylic acid at a concentration of 0.5% to 2%.

10. The skin lightening composition of claim 9, further comprising a permeation enhancer at a concentration of 2.5% to 7.5%.

11. The skin lightening composition of claim 10, wherein the permeation enhancer is diethylene glycol monoethyl ether.

12. The cosmetic composition of claim 8, further comprising from about 0.1 to about 5% by weight of one or more of azelaic acid, salicylic acid, lactic acid and glycolic acid.

13. The cosmetic composition of claim 8, further comprising from about 0.1 to about 5% by weight of one or more of 0.01 to 1% licorice extract and green tea extract.

14. A method of lightening a hyperpigmented condition of the skin, comprising:
topically applying a cosmetic composition comprising:
from about 0.1 to about 10% by weight a stable kojic acid derivative;
from about 0.1 to about 5% by weight retinoid:
from about 0.1 to about 5% by weight of a resorcinol derivative; and
a cosmetically acceptable vehicle.

15. The method of claim 14, wherein the stable kojic acid derivative is kojic dipalmitate at a concentration of 2.5% to 5%, the retinoid is retinol at a concentration of.5% to 2.5%; and the resorcinol derivative is 4-hexylresorcinol at a concentration of 0.75% to 2%.

16. The method of claim 15, wherein the cosmetic composition further comprises salicylic acid at a concentration of 0.5% to 2%.

17. The method of claim 15, wherein the cosmetic composition further comprises a permeation enhancer at a concentration of 2.5% to 7.5%.

18. The method of claim 15, wherein the cosmetic composition further comprises from about 0.1 to about 5% by weight of one or more of azelaic acid, salicylic acid, lactic acid and glycolic acid.

19. The method of claim 15, wherein the cosmetic composition further comprises from about 0.1 to about 5% by weight of one or more of 0.01 to 1% licorice extract and green tea extract.

20. The method of claim 15, wherein the skin condition is selected from the group consisting of solar lentigenes, melasma, chloasma, scars, freckles, and other local hyperpigmented regions of the skin.
